(19) United States
(12) Patent Application Publication
(10) Pub. No.: US 2019/0282257 A1
(43) Pub. Date: Sep. 19, 2019

(54) FOOT ABRASION ASSEMBLY

(71) Applicant: Greg Roberts, Bloomington, IL (US)

(72) Inventor: Greg Roberts, Bloomington, IL (US)

(21) Appl. No.: 15/921,341

(22) Filed: Mar. 14, 2018

(51) Int. Cl. A61B 17/32; A47K 3/00

(52) U.S. Cl. CPC A61B 17/32; A47K 3/002; A61B 2017/00747; A61B 2017/320004; A47K 2201/00

(57) ABSTRACT

A foot abrasion assembly for removing dead skin from feet includes a pad that may be positioned in a shower thereby facilitating the pad to be stood upon during bathing. A plurality of couplers is provided and each of the couplers is coupled to the pad. Each of the couplers releasably engages the shower to retain the pad in the shower. A sheet is removably attached to the pad thereby facilitating the sheet to be stood upon during bathing. The sheet is comprised of an abrasive material to abrade a bottom side of the user's feet for removal of dead skin.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to abrasion devices and more particularly pertains to a new abrasion device for removing dead skin from feet.

### BRIEF SUMMARY OF THE INVENTION

An embodiment of the disclosure meets the needs presented above by generally comprising a pad that may be positioned in a shower thereby facilitating the pad to be stood upon during bathing. A plurality of couplers is provided and each of the couplers is coupled to the pad. Each of the couplers releasably engages the shower to retain the pad in the shower. A sheet is removably attached to the pad thereby facilitating the sheet to be stood upon during bathing. The sheet is comprised of an abrasive material to abrade a bottom side of the user's feet for removal of dead skin.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective in-use view of a foot abrasion assembly according to an embodiment of the disclosure.

FIG. 2 is a front view of pad and sheets of an embodiment of the disclosure.

FIG. 3 is a bottom view of pad of an embodiment of the disclosure.

FIG. 4 is a cross sectional view taken along line 4-4 of FIG. 3 of an embodiment of the disclosure.

### DETAILED DESCRIPTION OF THE INVENTION

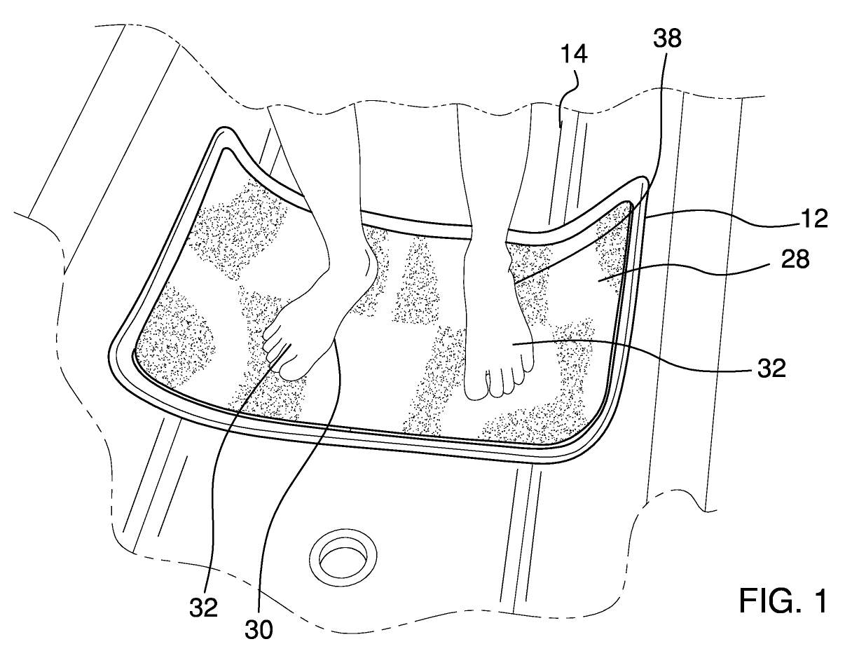

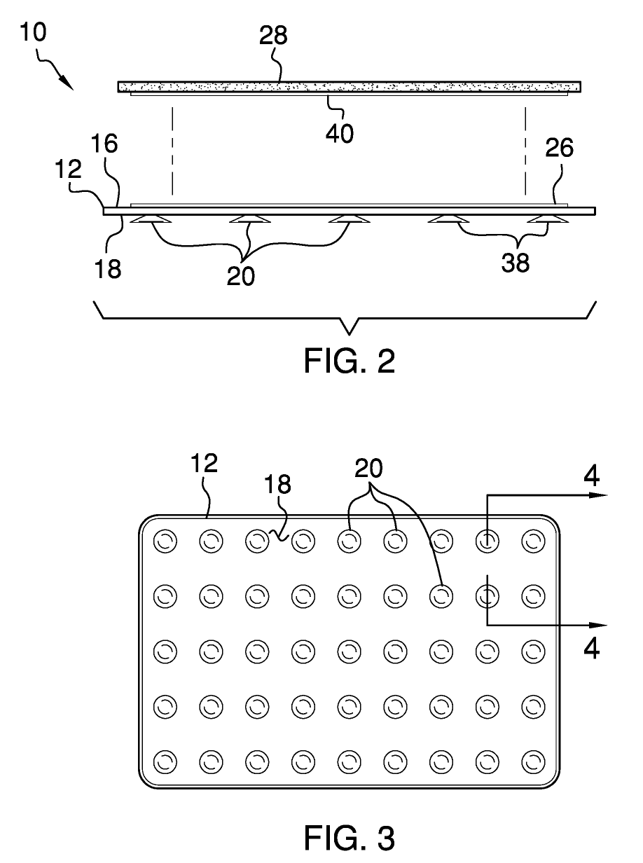

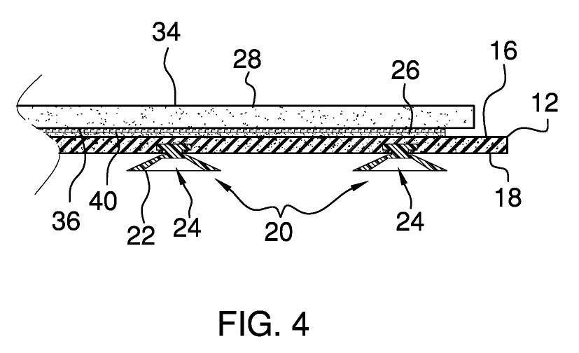

With reference now to the drawings, and in particular to FIGS. 1 through 4 thereof, a new abrasion device embodying the principles and concepts of an embodiment of the disclosure and generally designated by the reference numeral 10 will be described.

As best illustrated in FIGS. 1 through 4, the foot abrasion assembly 10 generally comprises a pad 12 that is positioned in the shower 14 thereby facilitating the pad 12 to be stood upon. The pad 12 has a top surface 16 and a bottom surface 18, and the pad 12 may be comprised of a fluid impermeable material such as rubber or the like. The shower 14 may be a bathing shower in a residence or the like. A plurality of couplers 20 is provided and each of the couplers 20 is coupled to the pad 12. Each of the couplers 20 releasably engages the shower 14 to retain the pad 12 in the shower 14. The pad 12 may have a width ranging between approximately 75.0 cm and 85.0 cm thereby facilitating the pad 12 to extend upwardly along sidewalls of the shower when the pad 12 is positioned in the shower.

Each of the couplers 20 is attached to the bottom surface 18 of the pad 12 and the couplers 20 are spaced apart from each other and are distributed on the bottom surface 18. Each of the couplers 20 has a distal end 22 with respect to the bottom surface 18. Moreover, each of the couplers 20 flares outwardly between the bottom surface 18 and the distal end 22 such that each of the couplers 20 defines a suction cup 24 for suctionally engaging the shower 14. A plurality of first mating members 26 is provided and each of the first mating members 26 is coupled to the top surface 16 of the pad 12. Each of the first mating members 26 may be a hook portion of a hook and loop fastener or a panel of water proof adhesive.

A sheet 28 is removably attached to the pad 12 thereby facilitating the sheet 28 to be stood upon. The sheet 28 is comprised of an abrasive material such as sand paper or the like. Thus, the sheet 28 abrades a bottom side 30 of the user's feet 32 for removal of dead skin. Moreover, water from the shower 14 softens the dead skin on the bottom side 30 of the user's feet 32 thereby enhancing removing the dead skin. The sheet 28 has an upper surface 34 and a lower surface 36, and the sheet 28 may have abrasion qualities similar to that of pumice stone or other similar, foot manicure tools. The sheet 28 may have a width that is equal to the width of the pad 12 thereby facilitating the sheet 28 to extend upwardly along the sidewalls of the shower 14. In this way the sheet 28 is positioned to facilitates outer sides 38 of the user's feet 32 to be abraded on the sheet 28.

A plurality of second mating members 40 is coupled to the lower surface 36 of the sheet 28. Each of the second mating members 40 releasably engages one of the first mating members 26 to retain the sheet 28 on the pad 12. Additionally, each of the second mating members 40 may be a loop portion of a hook and loop fastener or a panel of water proof adhesive.

In use, the pad 12 is positioned in the shower 14 such that each of the couplers 20 engages a floor of the shower 14. In this way the pad 12 is retained on the floor thereby facilitating the user to stand on the pad 12 during bathing. The user stands on the sheet 28 during bathing such that the water from the shower 14 has time to thoroughly soak the bottom side 30 of the user's feet 32. In this way the dead skin on the bottom side 30 of the user's feet 32 is softened. The user begins rubbing the user's feet 32 along the sheet 28 to abrade the dead skin from the bottom side 30 of the user's feet 32, especially the heel and the ball of the foot. Moreover, the shower 14 continually washes the dead skin away from the sheet 28 during the abrasion process. In this way general maintenance of the bottom side 30 of the feet 32 can be performed as well as removal of corns, warts and other skin maladies common to the bottom side 30 of feet 32.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A foot abrasion assembly being configured to be positioned in a shower thereby facilitating the user to stand thereon for abrading the user's feet, said assembly comprising:
a pad being positioned in the shower thereby facilitating said pad to be stood upon during bathing;
a plurality of couplers, each of said couplers being coupled to said pad, each of said couplers releasably engaging the shower to retain said pad in the shower; and
a sheet being removably attached to said pad thereby facilitating said sheet to be stood upon during bathing, said sheet being comprised of an abrasive material being exposed on said sheet wherein said sheet is configured to abrade a bottom side of the user's feet for removal of dead skin;
a first mating member being coupled to said pad; and
a second mating member being coupled to said sheet, said second mating member releasably engaging said first mating member to retain said sheet on said pad.

2. The assembly according to claim 1, wherein:
said pad has a top surface and a bottom surface; and
each of said couplers is attached to said bottom surface of said pad, said couplers being spaced apart from each other and being distributed on said bottom surface.

3. The assembly according to claim 2, wherein each of said couplers has a distal end with respect to said bottom surface, each of said couplers flaring outwardly between said bottom surface and said distal end such that each of said couplers defines a suction cup for suctionally engaging the shower.

4. The assembly according to claim 2, further comprising said first mating member being coupled to said top surface of said pad.

5. The assembly according to claim 4, wherein:
said sheet has an upper surface and a lower surface; and
said second mating member being coupled to said lower surface of said sheet.

6. A foot abrasion assembly being configured to be positioned in a shower thereby facilitating the user to stand thereon for abrading the user's feet, said assembly comprising:
a pad being positioned in the shower thereby facilitating said pad to be stood upon during bathing, said pad having a top surface and a bottom surface;
a plurality of couplers, each of said couplers being coupled to said pad, each of said couplers releasably engaging the shower to retain said pad in the shower, each of said couplers being attached to said bottom surface of said pad, said couplers being spaced apart from each other and being distributed on said bottom surface, each of said couplers having a distal end with respect to said bottom surface, each of said couplers flaring outwardly between said bottom surface and said distal end such that each of said couplers defines a suction cup for suctionally engaging the shower;
a first mating member being coupled to said top surface of said pad;
a sheet being removably attached to said pad thereby facilitating said sheet to be stood upon during bathing, said sheet being comprised of an abrasive material being exposed on said sheet wherein said sheet is configured to abrade a bottom side of the user's feet for removal of dead skin, said sheet having an upper surface and a lower surface; and
a second mating member being coupled to said lower surface of said sheet, said second mating member releasably engaging said first mating member to retain said sheet on said pad.
